(19) United States
(12) Patent Application Publication
(10) Pub. No.: US 2019/0175891 A1
(43) Pub. Date: Jun. 13, 2019

(54) INJECTION CATHETER AND METHOD OF USE THEREOF

(71) Applicant: COOK MEDICAL TECHNOLOGIES LLC, BLOOMINGTON, IN (US)

(72) Inventors: Brendan Schultz, Brighton, MI (US); Jeffry S. Melsheimer, Springville, IN (US)

(73) Assignee: COOK MEDICAL TECHNOLOGIES LLC, BLOOMINGTON, IN (US)

(21) Appl. No.: 16/190,359

(22) Filed: Nov. 14, 2018

Related U.S. Application Data
(60) Provisional application No. 62/595,651, filed on Dec. 7, 2017.

(51) Int. Cl. A61M 31/00; A61B 17/221; A61M 25/00

(52) U.S. Cl. CPC A61M 31/005; A61B 17/221; A61M 25/0043; A61M 27/008; A61B 2017/2212; A61F 2230/0093; A61M 25/0067

(57) ABSTRACT

One aspect of the invention provides an injection catheter system including an elongated sheath having a sheath lumen, extending from a distal end to a proximal end, and a catheter. In one embodiment the catheter includes an elongated member having a lumen and an expandable frame including a plurality of struts extending distally from the distal end of the elongated member and attaching to a tip at their distal ends. A graft material attaches to and extends between a proximal portion of the plurality of struts and to the elongated member, forming a collapsible funnel. Another aspect of the invention provides a method of using such a system for visualizing a target region within a vessel or organ of a subject.

### RELATED APPLICATIONS

This non-provisional patent application claims priority to U.S. Provisional Patent Application No. 62/595,651, filed Dec. 7, 2018, the contents of which are incorporated by reference in their entirety.

### TECHNICAL FIELD

The present invention relates generally to medical devices and more particularly to injection catheters used to deliver agents into a body cavity and to maintain such agents within the cavity for a required time period. In one embodiment the agent is a contrast agent, the presence of which allows visualization of a body organ or vessel. Certain aspects of the invention relate to methods of manufacturing and using such devices.

### BACKGROUND

Injection catheters are widely used in the medical profession for various intraluminal procedures. One example of such a procedure is retrograde pyelography. Retrograde pyelography is an urologic procedure in which the physician injects a radiocontrast agent into the ureter in order to visualize the ureter and kidney with fluoroscopy or radiography. The flow of contrast, up from the bladder to the kidney, is opposite the usual outbound flow of urine. Reasons for performing a retrograde pyelogram include identification of filling defects (e.g. stones or tumors), as an adjunct during the placement of ureteral stents or ureteroscopy, or to delineate renal anatomy in preparation for surgery.

Retrograde pyelography is generally performed when an intravenous excretory study (intravenous pyelogram or contrast CT scan) cannot be performed because of renal disease or allergy to intravenous contrast. Relative contraindications include the presence of infected urine, pregnancy and contrast allergy.

The procedure requires cystoscopy and the placement of a small tube into the lower part of the ureter to inject contrast and opacify the ureter and renal pelvis. Fluoroscopy or X-ray imaging are typically used for visualization. The procedure is usually performed under general or regional anesthesia.

When performing percutaneous kidney stone removal, the process of flushing the kidney with contrast media to identify the location of stones through fluoroscopy requires that the urinary system be temporarily occluded to prevent drainage of the contrast media. The urinary system may be occluded, for example, at the exit of the kidney or bladder, while the contrast media is injected. However, such an occlusion may present a number of problems. Many existing occlusion/injection devices have bulky tips and may cause irritation. Existing devices also may only have one occlusion diameter per device, requiring that the size of the device to be matched with the size of the region to be occluded. Many current devices are also prone to slippage if the application pressure on the device varies.

### SUMMARY

One aspect of the present invention provides an injection catheter system including an elongated sheath having a sheath lumen extending from a distal end to a proximal end. The system also includes a catheter, at least a distal portion of which may be positioned within the sheath lumen.

In one embodiment, the catheter includes an elongated member having an lumen extending therethrough along a longitudinal axis from a proximal end to a distal end. An expandable frame having a laterally expanded configuration and a collapsed configuration is attached to a distal end of the elongated member. In one embodiment, the frame includes a plurality of struts extending distally from the distal end of the elongated member. Each strut has a proximal end attaching to the distal end of the elongated member, a central portion and a distal end. A tip attaches to a distal end of each of the struts. The tip may have an atraumatic distal portion.

A graft material (membrane) attaches to and extends between a proximal portion of the struts and to the elongated member, and forms a collapsible funnel. The expandable frame is biased towards a laterally expanded configuration in which the funnel forms a laterally extended exit to the distal end of the lumen of the elongated member. When the expandable frame is in a collapsed state, the distal portion of the catheter is slidably positionable within the sheath lumen.

In one embodiment, the struts include a polymer, a stainless steel or a memory nickel-titanium alloy. In another embodiment, the graft is impermeable. In yet another embodiment, the graft includes a nylon, polyolefin, polyester, polyurethane, fluoropolymer, polyethylene, polytetrafluoroethylene, latex, rubber, or mixtures of at least two of these materials

In another embodiment, a fluid valve in positioned in-line with the lumen of the elongated member near the proximal end of the elongated member. A luer fitting may be present proximal of the fluid valve. In yet another embodiment, the sheath and/or the catheter include a radiopaque marker and/or an echogenic marker.

In other embodiments, when the collapsible funnel is in a fully expanded state, it may have a maximum lateral dimension of less than four times, three times, or two times the maximum outside dimension of the distal portion of the elongated sheath.

Another aspect of the present invention provides a method of visualizing an interior portion of a vessel or organ lumen of a subject, for example, the urinary system. One embodiment of this method includes introducing an injection catheter system as disclosed herein into the urinary system of the patent. The distal end of the elongated sheath is positioned at the target local within the interior portion and the expandable frame extended from the distal end of the sheath, whereby the expandable frame assumes a laterally expanded configuration (i.e. away from the longitudinal axis) to restrict the vessel or organ lumen. If necessary, the catheter is then retracted proximally to restrict the urinary system proximally of the target location. A contrast medium is then introduced to a position distal of the collapsible funnel and the target location of the subject's urinary system visualized. In one embodiment, the contrast medium is introduced into the funnel through the elongated member lumen. The interior portion of the urinary system may be a vestibule of a kidney. In yet another embodiment, the injection catheter system is introduced into the urinary system over a wire guide.

### BRIEF DESCRIPTION OF THE DRAWINGS

FIG. 1 is a schematic view of a distal portion of an injection catheter according to one embodiment of the present invention.

FIG. 2 is a schematic view of a distal portion of an injection catheter according to one embodiment of the present invention. The catheter is positioned to deliver contrast fluid to a kidney lumen while blocking the junction between the lumen of the kidney and the ureter.

FIGS. 3(A-C) are schematic illustrations showing the collapsible frame according to one embodiment of the present invention either fully expanded, FIG. 3(A), partially constrained, FIG. 3(B), and fully constrained, FIG. 3(C).

### DETAILED DESCRIPTION OF THE PREFERRED EMBODIMENTS

### Definitions

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety.

The uses of the terms “a” and “an” and “the” and similar references in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”) provided herein, is intended merely to better illuminate the invention and does not pose a limitation on the scope of the invention unless otherwise claimed. No language in the specification should be construed as indicating any non-claimed element as essential to the practice of the invention.

As used herein the terms “comprise(s),” “include(s),” “having,” “has,” “can,” “contain(s),” and variants thereof, are intended to be open-ended transitional phrases, terms, or words that do not preclude the possibility of additional acts or structures. The present invention also contemplates other embodiments “comprising,” “consisting of” and “consisting essentially of,” the embodiments or elements presented herein, whether explicitly set forth or not.

As used herein, the term “implantable” refers to an ability of a medical device to be positioned at a location within a body, such as within a body vessel. Furthermore, the terms “implantation” and “implanted” refer to the positioning of a medical device at a location within a body, such as within a body vessel or organ.

As used in the specification, the terms “proximal” and “distal” should be understood as being in the terms of a physician using the device. The term distal means the portion of the device, or component, which is first implanted within the body of a subject or which is farthest from the physician and the term proximal means the portion of the device which is nearest to the physician.

As used herein, the term “body lumen” means any body lumen or lumen, including but not limited to vascular coronary or peripheral lumens, esophageal, intestinal, biliary, urethral and ureteral passages, and interior lumens of the heart, kidney and bladder.

### Injection Catheter System

For the purpose of promoting an understanding of the principles of the invention, reference will now be made to embodiments, some of which are illustrated in the drawings, and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

One aspect of the present invention provides an injection catheter system for use in delivering a contrast or other medium to vessel or organ lumen of a human or veterinary subject. During such procedures, it is often required that the vessel or exit from the organ lumen is temporary blocked. For example, it may be necessary to block the vessel or exit to prevent drainage of a contrast medium while a target region containing the contrast medium is visualized using fluoroscopy or radiography. The present embodiments include an implantable injection catheter system that may be used to deliver contrast medium to the required region and also to temporarily block the vessel or organ lumen at a region proximal to the target location to prevent drainage of the medium.

Embodiments of the system will now be described with particular reference to retrograde pyelography, a urologic procedure where the physician injects a radiocontrast agent into the ureter in order to visualize the ureter and/or kidney with fluoroscopy or radiography. During this procedure, the ureter of ureter/kidney junction is temporarily blocked at a region proximal of the target region to be visualized. For example, the junction between the lumen of the kidney and the ureter may be blocked to allow for visualization of the kidney lumen.

However, although the use of the catheter system is illustrated with reference to retrograde pyelography, the present invention includes embodiments where this system is utilized to visualize another region of the urinary system, for example, the bladder, of another organ or vessel. For example, the catheter system may be utilized to visualize vascular coronary or peripheral lumens, esophageal, intestinal and biliary passages, and interior lumens of the heart.

One embodiment of the injection catheter system includes an elongated sheath having an interior lumen and a catheter having a distal portion sized to fit into and slide within the lumen of the sheath. The catheter includes an elongated member having a lumen extending from its proximal end to its distal end and an expandable frame attached to its distal end. In one embodiment, the expandable frame includes a number of struts, each having first end attached to the distal end of the elongated member and a second end attached to a tip forming the distal end of the catheter. The tip may include an atraumatic distal portion. Preferably, a distal portion of the catheter, including the elongated member and expandable frame, is sized and shaped to fit within the lumen of the sheath with the expandable frame in a collapsed configuration and is free to slide sufficiently within the lumen to allow the expandable frame to be either contained within the lumen (in a collapsed configuration) or to extend from the distal end of the elongated member (in an expanded configuration).

Preferably, the expandable frame is biased towards an expanded configuration where the frame extends laterally (away from) the longitudinal axis of the catheter unless constrained (for example, by the elongated sheath. For example, in one embodiment, the struts are biased towards a curved shape where, unless constrained, the central portion of the strut extends laterally from the longitudinal axis of the catheter. In this state, the frame, in its expanded configuration, forms an enlarged region at the distal end of the catheter.

In other embodiments, the expandable frame includes cross members linking between the longitudinal struts or is a braided structure attaching to the elongated member and the tip. For example, the frame may be formed from a number of braided (interlaced) filaments, the ends of which filaments are gathered together at the distal end of the elongated member and at the tip.

The expandable frame may be formed from, for example, stainless steel that is biased (spring loaded) in a curved state, or a memory shape nickel-titanium alloy, such as NITINOL. In other embodiments, the frame is formed from, or includes, gold, tantalum, platinum or platinum iridium, niobium, tungsten, inconel, ceramic, nickel, titanium, stainless steel/titanium composite, cobalt, chromium, cobalt/chromium alloys, magnesium, aluminum, or other biocompatible metals and/or composites or alloys such as carbon or carbon fiber.

In yet other embodiments, the struts may be formed from a polymer material, such as but not limited to, cellulose acetate, cellulose nitrate, cross-linked polyvinyl alcohol (PVA) hydrogel, cross-linked PVA hydrogel foam, polyurethane, styrene isobutylene-styrene block copolymer (Kraton), polyethylene terephthalate, polyurethane, polyamide, polyester, polyorthoester, polyanhydride, polyether sulfone, polycarbonate, polypropylene, high molecular weight polyethylene, polytetrafluoroethylene, or other biocompatible polymeric material, or mixture of copolymers thereof.

Unless constrained, the frame forms a laterally expanded region at the distal end of the catheter. In one embodiment, the proximal and distal ends of the struts are positioned along the longitudinal axis of the catheter while the central portions of the struts are positioned laterally from the longitudinal axis. However, when the catheter is positioned within the lumen of the sheath, the central portions of the struts are constrained by the inner wall of the sheath, forcing the frame to an collapsed configuration.

A graft material attaches to and extends between a proximal portion of the struts and to the elongated member forming a funnel-shaped enclosure when the frame is expanded. In this configuration, the distal end of the lumen extending through the elongated member provides fluid access to the interior of the funnel. Preferably, the graft is formed from an impermeable material such that the funnel may be filled by delivery of fluid through the lumen of the elongated member. When the catheter, including the frame, is positioned within the lumen of the sheath, the funnel collapses and the graft material folds adjacent to the proximal region of the struts. The graft may be formed from, or include, a polymer material such as, but not limited to, a nylon, polyolefin, polyethylene terephthalate, polyester, polyurethane, fluoropolymer, polyethylene, polytetrafluoroethylene, latex, rubber, or mixtures of at least two of these materials.

In one embodiment, the graft extends from the distal end of the elongated member to the central region of the struts, i.e. to the region of greatest lateral expansion in an unconstrained configuration. In other embodiments the graft extends only part way to this region or distal of this region. When the graft is to be used to block a vessel on constant cross-section, preferably the graft will extend at least to the region of greatest lateral dimension. However, if the funnel is to be positioned where the cross-section dimension increases in a distal direction, for example, at the ureter-kidney lumen junction, it may be necessary that the graft extends to the region of maximum dimension in order to effectively block drainage of the medium from the kidney lumen. The distal end of the frame is preferably free of the graft material or an impermeable fluid seal so that contrast medium delivered through the lumen of the elongated member is free to flood the region distal of the catheter to allow for visualization of this region.

The lateral expansion of the graft when the expandable frame is in the full expanded configuration should be sufficient to block the vessel or organ lumen to be visualized at a position just proximal of the vessel or organ lumen. In certain embodiments, the graft (collapsible funnel) has a maximum lateral dimension of less than five times, four times, three times, or two times, the maximum outside dimension of the elongated sheath.

In one embodiment, a fluid valve is positioned at the proximal end of the catheter, in-line with the lumen of the elongated member. A luer fitting may be positioned proximal of the valve, if present, at the proximal end of the catheter. In other embodiments, the catheter and/or the sheath may include a radiopaque marker and/or echogenic marker to allow for visualization of the system during placement.

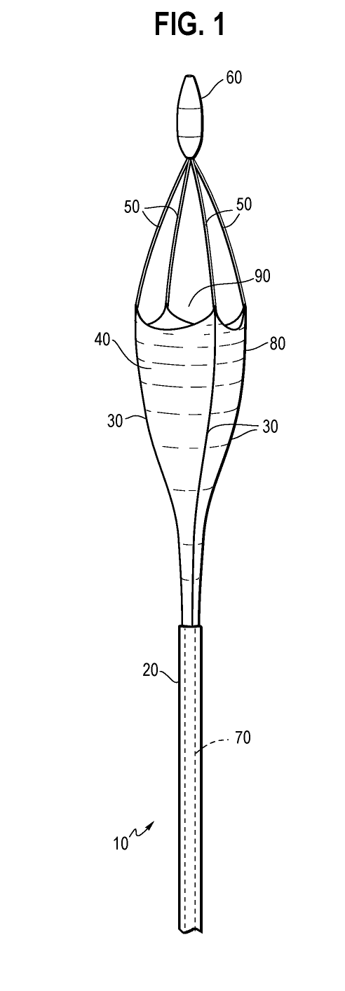

Turning now to FIG. 1, there is here illustrated a schematic representation of the distal end of a catheter according to one embodiment of a device of the present invention. Catheter 10 includes elongated member 30 having a lumen 70 extending to its distal end. Expandable frame 80 is positioned at the distal end of elongated member 20 includes a number of struts each attaching to elongated member 20 and extending to distal tip 60. Proximal portions 30 of the struts attach to graft material 40, which forms a collapsible funnel 90. The graft material is sealed to the distal region of elongated member 20 allowing fluid delivered through lumen 70 to enter and fill the interior of the funnel and for the funnel, when expanded, to prevent drainage of the fluid in a proximal direction. Distal portions 50 of the struts are not covered by the graft material. Thus, the contrast medium is free to escape from the top of the funnel and to fill a portion of the vessel or organ lumen just distal of the funnel.

### Method of Visualizing an Interior Portion of a Vessel or Organ Lumen

Another aspect of the present invention provides a method of visualizing an interior portion of a vessel or organ lumen of a human or veterinary subject. In one embodiment, the method includes delivering a contrast medium to the vessel or region and temporarily blocking drainage of the contrast medium.

In one embodiment, an injection catheter system as disclosed herein is implanted within the vessel or organ lumen, optionally over a wire guide. The injection catheter is advanced to position the distal end of the sheath at the required position. The catheter is positioned within the lumen of the sheath with the expandable frame in a collapsed configuration either before or after implantation and delivery of the sheath to the required position.

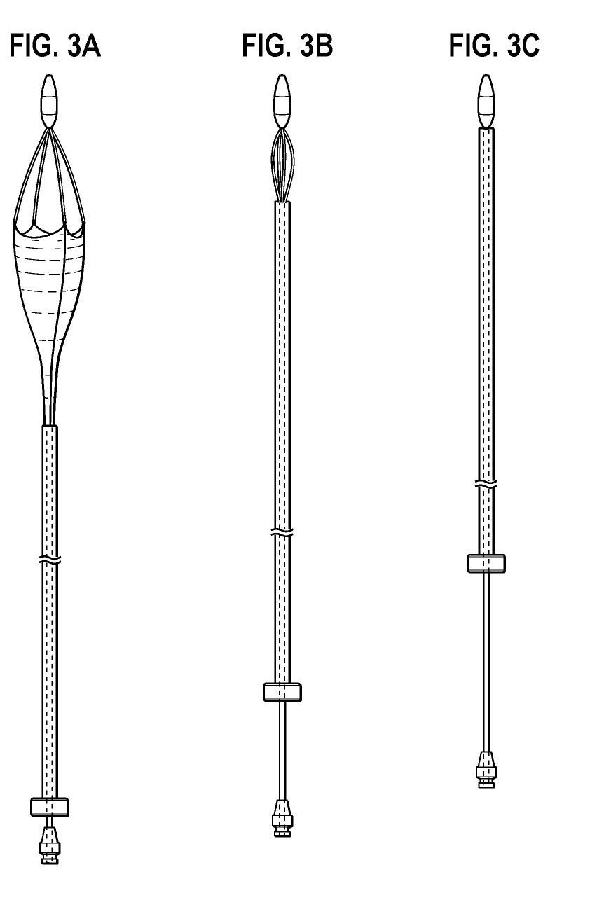

The expandable frame is advanced out of the distal end of the sheath, causing the frame to expand as it is no longer constrained to the sheath. The frame may be advanced out of the sheath either by moving the sheath proximally while maintaining the catheter in position or by moving the proximal end of the catheter distally while maintaining the sheath in position. FIGS. 3(A-C) are schematic illustrations showing the collapsible frame either fully expanded, FIG. 3(A), partially constrained, FIG. 3(B), or fully constrained, FIG. 3(C). Preferably, the dimensions of the distal end of the funnel are such that expansion of the frame results in the impermeable graft material forming the funnel blocking the vessel or organ lumen at a position just proximal from the target region to be visualized.

Contrast medium is then introduced into the interior of the funnel through the lumen of the elongated member. Sufficient contrast medium is utilized such that the vessel or organ lumen proximal of the funnel is flooded with the medium. The region under investigation is then visualized using, for example, fluoroscopy or X-ray imaging. After the required examination is completed the contrast medium may be drained from the vessel or organ through the lumen of the elongated member and the frame collapsed by drawing it into the lumen of the sheath. The sheath may then be removed from the body of the subject.

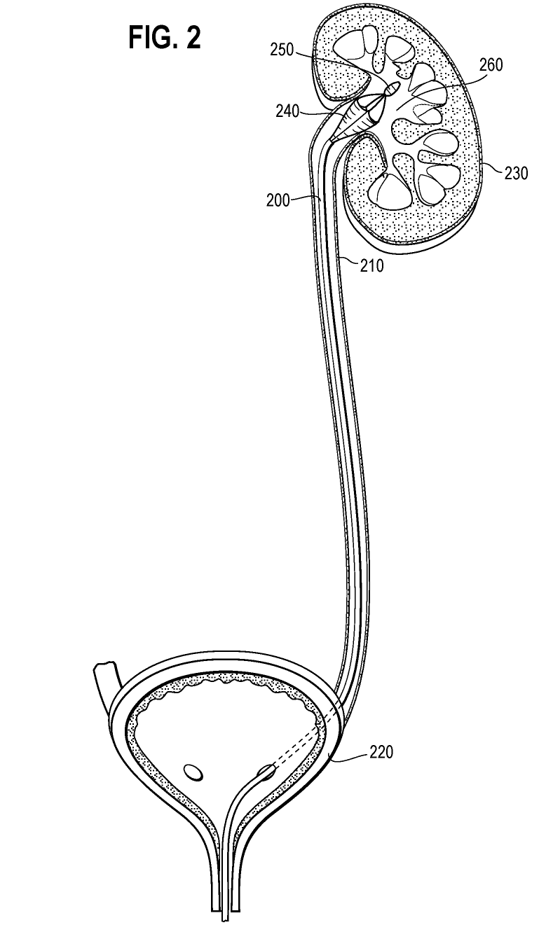

Turning now to FIG. 2, there is here illustrated one embodiment of an injection catheter of the present invention position to allow for visualization of the kidney of a subject. For clarity, the sheath is not illustrated in the figure. Here, elongated member 200 is shown threaded through the bladder 200 and ureter 210 to position expandable frame 240 at the junction of the ureter 200 and vestibule 260 of kidney 230. The frame is illustrated in an expanded configuration with distal tip 250 positioned with in the kidney. The proximal region of the expandable frame is shown in position to block flow of fluid out of the kidney into the ureter.

Although the invention has been described and illustrated with reference to specific illustrative embodiments thereof, it is not intended that the invention be limited to those illustrative embodiments. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

What is claimed is:

1. An injection catheter system comprising:
an elongated sheath having a sheath lumen extending from a distal end to a proximal end of the elongated sheath, and
a catheter comprising:
an elongated member having an elongated member lumen extending along a longitudinal axis from a proximal end to a distal end of the elongated member;
an expandable frame having a laterally expanded configuration and a collapsed configuration and comprising a plurality of struts extending distally from the distal end of the elongated member, each strut having a proximal end attaching to from the distal end of the elongated member, a central portion and a distal end;
a tip attaching to a distal end of each of the plurality of struts; and
a graft attaching to and extending between a proximal portion of the plurality of struts and to the elongated member, wherein the graft forms a collapsible funnel,
wherein the expandable frame is biased towards a laterally expanded configuration and wherein the catheter is slidably positionable within the sheath lumen with the expandable frame in the collapsed configuration.

2. The injection catheter system of claim 1, wherein the expandable frame is slidably positionable within the sheath lumen in a collapsed configuration whereby the central portions of the plurality of struts are constrained towards the longitudinal axis.

3. The injection catheter system of claim 1, wherein the plurality of struts comprises a material selected from the group consisting of a polymer, a stainless steel or a memory nickel-titanium alloy.

4. The injection catheter system of claim 1, wherein the plurality of struts comprises a stainless steel or a memory nickel-titanium alloy.

5. The injection catheter system of claim 1, wherein the plurality of struts comprises a polymer.

6. The injection catheter system of claim 1, wherein the graft is impermeable.

7. The injection catheter system of claim 6, wherein the graft comprises a material selected from the group consisting of a nylon, polyolefin, polyester, polyurethane, fluoropolymer, polyethylene, polytetrafluoroethylene, latex, rubber, and mixtures thereof.

8. The injection catheter system of claim 1, further comprising a fluid valve at the proximal end of the elongated member lumen.

9. The injection catheter system of claim 1, wherein the elongated sheath comprises at least one of a radiopaque marker and an echogenic marker.

10. The injection catheter system of claim 1, wherein the elongated member comprises at least one of a radiopaque marker and an echogenic marker.

11. The injection catheter system of claim 1, further comprising a luer fitting positioned proximal of the fluid valve.

12. The injection catheter system of claim 1, wherein, when in a fully expanded state, the collapsible funnel has a maximum lateral dimension of less than 4 times a maximum outside dimension of the elongated sheath.

13. The injection catheter system of claim 12, wherein, when in a fully expanded state, the collapsible funnel has a maximum lateral dimension of less than 3 times a maximum outside dimension of the elongated sheath.

14. The injection catheter system of claim 12, wherein, when in a fully expanded state, the collapsible funnel has a maximum lateral dimension of less than 2 times a maximum outside dimension of the elongated sheath.

15. The injection catheter system of claim 1, wherein the tip comprises an atraumatic distal portion.

16. A method of visualizing an interior portion of a subject's urinary system, comprising:
introducing an injection catheter system into the urinary system, the injection catheter system comprising:
an elongated sheath comprising a sheath lumen and a catheter positionable within the sheath lumen, the catheter comprising:
an elongated member having an elongated member lumen extending along a longitudinal axis from a proximal end to a distal end of the elongated member;
an expandable frame having a laterally expanded configuration and a collapsed configuration and comprising a plurality of struts extending distally from the distal end of the elongated member, each strut having a proximal end attaching to from the distal end of the elongated member, a central portion and a distal end;
a tip attaching to a distal end of each of the plurality of struts; and
a graft attaching to and extending between a proximal portion of the plurality of struts and to the distal end of the elongated member,
wherein the graft forms a collapsible funnel,
wherein the expandable frame is biased towards a laterally expanded configuration and wherein the catheter is slidably positionable within the sheath lumen with the expandable frame in the collapsed configuration;
positioning a distal end of the elongated sheath at the interior portion;
positioning the expandable frame distally of the distal end of the elongated sheath, whereby the expandable frame assumes the laterally expanded configuration;
retracting the catheter proximally whereby the urinary system is restricted proximally of the interior portion;
introducing a contrast medium to a position distal of the collapsible funnel; and
visualizing the contrast medium at the interior portion of the subject's urinary system.

17. The method of claim 16, wherein the interior portion of the urinary system is a vestibule of a kidney.

18. The method of claim 16, wherein the injection catheter system is introduced into the urinary system over a wire guide.

19. The method of claim 16, wherein the contrast medium is introduced through the elongated member lumen.

20. The method of claim 16, wherein positioning the expandable frame distally of the distal end of the elongated sheath comprises retracting the elongated sheath proximally.
